(19) United States
(12) Patent Application Publication
(10) Pub. No.: US 2013/0316386 A1
(43) Pub. Date: Nov. 28, 2013

(54) TEST ELEMENT, TEST KIT, TEST DEVICE, AND TEST METHOD

(71) Applicant: Kabushiki Kaisha Toshiba, Minato-ku (JP)

(72) Inventors: Ichiro Tono, Kanagawa-ken (JP); Takaaki Wada, Tokyo (JP); Kayoko Oomiya, Kanagawa-ken (JP); Shingo Kasai, Kanagawa-ken (JP); Masaaki Hirakawa, Kanagawa-ken (JP)

(73) Assignee: Kabushiki Kaisha Toshiba, Minato-ku (JP)

(21) Appl. No.: 13/956,911

(22) Filed: Aug. 1, 2013

Related U.S. Application Data
(62) Division of application No. 13/207,690, filed on Aug. 11, 2011.

(30) Foreign Application Priority Data
Sep. 10, 2010 (JP) 2010-203118

(51) Int. Cl. C12Q 1/28; C12Q 1/26

(52) U.S. Cl. CPC C12Q 1/28; C12Q 1/26
USPC 435/28; 435/288.7; 435/25

(57) ABSTRACT

According to one embodiment, a test element includes a base, a pair of optical element units, an optical waveguide unit, a detection unit and a holding unit. The base has transparency. The pair of optical element units are arranged away from each other on a major surface of the base. The optical waveguide unit is provided on the major surface of the base. The detection unit is provided on a major surface of the optical waveguide unit of between the optical element units. The major surface of the optical waveguide unit is an opposite side which touches the base. The holding unit is in a frame shape, and one end of the holding unit being is provided to protrude from a major surface of the detection unit. The detection unit includes a color former and a film-formed body holding the color former.

### CROSS-REFERENCE TO RELATED APPLICATIONS

This application is a division of and claims the benefit of priority under 35 U.S.C. §120 from U.S. Ser. No. 13/207,690 filed Aug. 11, 2011, and claims the benefit of priority under U.S.C. §119 from Japanese Patent Application No. 2010-203118 filed Sep. 10, 2010, the entire contents of each of which are incorporated herein by reference.

### FIELD

Embodiments described herein relate generally to a test element, a test kit, a test device, and a test method.

### BACKGROUND

A test element is known having a configuration in which a color development unit, a reflection unit, a reaction unit, and a development unit are sequentially stacked on a transparent sheet.

In such a test element, a sample liquid is introduced into the reaction unit containing reagents in a dry state to react the sample liquid with the reagents; the resulting reaction product is introduced into a color former, which causes an absorbance change; and the magnitude of the absorbance change is measured.

In this test element, in those cases where the measuring object is an enzyme and the activity thereof is quantified, it is necessary to put a plurality of reagents in large amounts in the reaction unit. This may cause the formation of scatterers, resulting in a large measurement error in the magnitude of the absorbance change, and may slow down the diffusion rate of the reaction product and the like, leading to a long test time.

### BRIEF DESCRIPTION OF THE DRAWINGS

FIG. 1 is a schematic cross-sectional view for illustrating a test element according to the embodiment;

FIG. 2 is a schematic diagram for illustrating the way of a reaction;

FIG. 3 is a schematic graph for illustrating the change over time of the absorbance;

FIG. 4 is a schematic graph for illustrating the relationship between the enzyme activity (ALT (GPT) activity) and the magnitude of the absorbance change; and

FIG. 5 is a schematic view for illustrating a test device according to the embodiment.

### DETAILED DESCRIPTION

In general, according to one embodiment, a test element includes a base, a pair of optical element units, an optical waveguide unit, a detection unit and a holding unit. The base has transparency. The pair of optical element units are arranged away from each other on a major surface of the base. The optical waveguide unit is provided on the major surface of the base. The detection unit is provided on a major surface of the optical waveguide unit of between the optical element units. The major surface of the optical waveguide unit is an opposite side which touches the base. The holding unit is in a frame shape, and one end of the holding unit being is provided to protrude from a major surface of the detection unit. The detection unit includes a color former and a film-formed body holding the color former.

In general, according to one embodiment, a test kit includes the above test element and a test solution containing a substrate for an enzyme to be measured and a buffer solution.

In general, according to one embodiment, a test kit includes the above test element, a substrate for an enzyme to be measured, and a buffer solution.

In general, according to one embodiment, a test device includes a light sending unit and_a light receiving unit. The light sending unit sends light into one of a pair of optical element units provided in the above test element. The light receiving unit receives light from another one of the pair of optical element units and converts the light into an electric signal in accordance with an intensity of the light.

In general, according to one embodiment, a test method is disclosed. The method can include mixing at least a substrate for an enzyme to be measured, a buffer solution, and a sample liquid containing the enzyme. The method can include supplying the mixed liquid in a prescribed amount into a holding unit provided in the above test element. Finding a magnitude of an absorbance change caused by color development of a color former provided in a detection unit of the test element. In addition, the method can include finding an enzyme activity based on the magnitude of the absorbance change.

Various embodiments will be described hereinafter with reference to the accompanying drawings. In the drawings, like components are marked with the same reference numerals and a detailed description is omitted as appropriate.

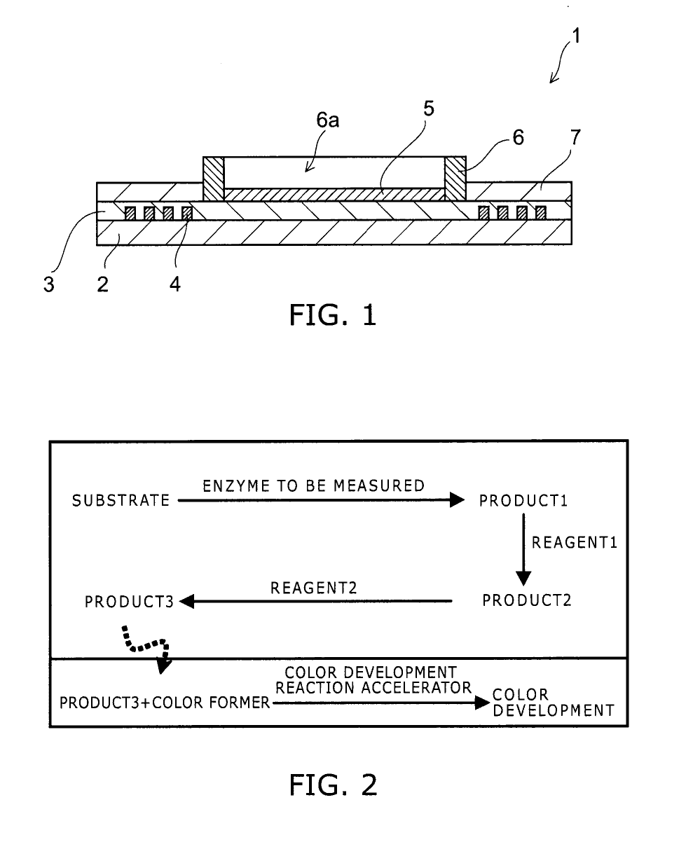

FIG. 1 is a schematic cross-sectional view for illustrating a test element according to the embodiment.

As shown in FIG. 1, a test element 1 includes a base 2, an optical waveguide unit 3, optical element units 4, a detection unit 5, a holding unit 6, and a protection unit 7.

The base 2 is in a flat plate shape and is made of a translucent material. The base 2 may be made of, for example, glass (e.g. alkali-free glass), quartz, or the like. The optical waveguide unit 3 has a higher refractive index than the base 2, and is provided on one major surface of the base 2. That is, the optical waveguide unit 3 is provided so as to cover the major surface of the base 2 and a pair of optical element units 4 described later. The optical waveguide unit 3 may be made of a polymer resin or the like, and may be in a film form with a nearly uniform thickness set in a range from 3 μm to 300 μm.

The optical element unit 4 is provided in a pair near to both ends of the major surface of the base 2 on the side on which the optical waveguide unit 3 is provided. That is, a pair of optical element units 4 are arranged away from each other on the major surface of the base 2. The optical element unit 4 illustrated in FIG. 1 functions as a diffraction grating when light is sent to and emitted from the optical waveguide unit 3. Accordingly, the optical element unit 4 has a higher refractive index than the base 2, and is provided in a lattice configuration with a prescribed pitch dimension. For example, the optical element unit 4 may be provided in a lattice configuration with a pitch dimension of 1 μm. The pitch dimension, however, is not limited thereto but may be altered as appropriate.

The optical element unit 4 may be made of, for example, titanium oxide (TiO₂), tin oxide (SnO₂), zinc oxide, lithium niobate, gallium arsenic (GaAs), indium tin oxide (ITO), polyimide, or the like. Although the optical element unit 4 functioning as a diffraction grating is illustrated, an optical element capable of causing light to enter the optical waveguide unit 3 may be appropriately selected. For example, the optical element unit may be what functions as a prism when light is sent to and emitted from the optical waveguide unit 3.

The detection unit 5 is in a film form, and is provided on a central portion of a major surface of the optical waveguide unit 3 on the side opposite to the side on which the base 2 is provided. That is, the detection unit 5 is provided on the major surface of the optical waveguide unit 3 of between the optical element units 4. The major surface of the optical waveguide unit 3 is an opposite side which touches the base 2. The detection unit 5 may include a color former and a film-formed body holding the color former.

The film-formed body may have a porous structure, and may be configured to hold the color former in voids of the porous structure, for example. Examples of the material of the film-formed body include carboxymethyl cellulose (CMC), polyethylene glycol (PEG), and the like.

The detection unit 5 can be formed by, for example, mixing the color former, a polymer for film-formation, and the like homogeneously with a mixed solvent of water and an aqueous organic solvent such as alcohol to make a precursor solution, and drying the precursor solution into a film form.

In the case where a peroxide such as hydrogen peroxide is produced as a reaction product, the color former contained in the detection unit 5 may be a benzidine-based color former. Examples of the benzidine-based color former include 4-chloro-1-naphthol, 3,3′-diaminobenzidine, 3,3′,5,5′-tetramethylbenzidine (hereinafter, may be referred to as TMBZ), a hydrochloride of TMBZ (3,3′,5,5′-tetramethylbenzidine.2HCl.2H₂O), and the like. In this case, it is possible to use TMBZ which has low solubility in water and very low harmfulness to a living body.

In addition, in the case where NADH (reduced nicotinamide adenine dinucleotide) or the like is produced as the reaction product, the color former contained in the detection unit 5 may be a tetrazolium salt or the like.

Here, if the detection unit is formed of the film-formed body, the color former, and reagents, a test can be performed only by supplying a sample liquid to the detection unit.

In this case, in those reaction series in which an enzyme activity is quantified, it is necessary to quantify an end product produced through two or more elementary reactions

Accordingly, a plurality of reagents are used at high concentrations, and it is necessary to put the plurality of reagents in the detection unit.

However, it is difficult to put a plurality of reagents in the detection unit.

This is because, even if it is possible to put a plurality of reagents in the detection unit, the large amount of the reagents may cause crystallization and/or precipitation of reagents to form scatterers. The formation of scatterers may lead to a large measurement error in the measurement of the magnitude of an absorbance change described later.

Furthermore, in such a detection unit, the water originally contained in the sample liquid serves as a reaction field, resulting in the progress of elementary reactions. In such a case, it is expected that the diffusion rate of the reaction product changing with the viscosity of a solvent and the like will affect and change the reaction rate, and that the reaction rate for the total plurality of elementary reactions will be not a little affected by differences between individual samples. This may slow down the diffusion rate of the reaction product and lead to a long test time. Furthermore, if the detection unit is configured to contain the color former and a plurality of reagents, the test element becomes large.

Therefore, in the embodiment, the detection unit 5 is formed of the color former and the film-formed body holding the color former. Furthermore, a configuration is employed in which a substrate for an enzyme to be measured and reagents used during performing elementary reactions are put in a test solution described later and the test solution is used as a reaction field. Details of the test solution are described later.

The detection unit 5 may be also formed of the film-formed body, the color former, and a color development reaction accelerator. That is, also a configuration is possible in which the detection unit 5 further contains the color development reaction accelerator and the film-formed body holds the color former and the color development reaction accelerator. In this case, although the color development reaction accelerator is added in addition to the color former, the amount of the color development reaction accelerator is small as compared to the case where a plurality of reagents are put in the detection unit. Thereby, the occurrence of the problem described above can be suppressed. However, preferably the activity of the color development reaction accelerator does not decrease when the color development reaction accelerator is dissolved in the precursor solution used during forming the detection unit 5.

In the case where a peroxide such as hydrogen peroxide is produced as the reaction product, a peroxidase (POD) and the like, for example, may be used as the color development reaction accelerator.

In the case where NADH (reduced nicotinamide adenine dinucleotide) or the like is produced as the reaction product, diaphorase and the like, for example, may be used as the color development reaction accelerator.

The color former and the color development reaction accelerator are not limited those illustrated but may be appropriately altered in accordance with the measuring object.

In this case, the amount of the test solution held on the upper side of the detection unit 5 is large as compared to the case where the water originally contained in a sample liquid is used as a reaction field. Therefore, the holding unit 6 is provided, and a prescribed amount of the test solution can thereby be held on the upper side of the detection unit 5.

The holding unit 6 is in a frame shape, and is provided so as to surround the detection unit 5. One end of the holding unit 6 is provided on the major surface of the optical waveguide unit 3 in a liquid-tight manner, and the other end is provide so as to protrude from the major surface of the detection unit 5. Therefore, a holding space 6a holding the test solution described later is formed on the upper side of the detection unit 5.

In this case, the volume of the test solution held by the holding unit 6 is preferably set not less than a prescribed value. According to the findings obtained by the inventors, the volume of the test solution held by the holding unit 6 is preferably set not less than 10 μL (microliters).

Hence, the volume of the holding space 6a may be set not less than 10 μL (microliters). That is, one end of the holding unit 6 protrudes from the major surface of the detection unit 5 so that a space (the holding space 6a) with a volume of not less than 10 μL may be formed on the upper side of the detection unit 5.

The protection unit 7 is provided so as to cover both end portions of the optical waveguide unit 3 that are opposed to regions where the optical element units 4 are formed. That is, the protection unit 7 is provided so as to cover the major surface of the optical waveguide unit 3 on the outside of the holding unit 6.

The protection unit 7 is made of a material with a lower refractive index than the optical waveguide unit 3. Furthermore, the protection unit 7 is made of a material with a high resistance to the liquid held by the holding unit 6. The protection unit 7 may be made of, for example, a fluororesin or the like.

According to the embodiment, a substrate for an enzyme to be measured and reagents are put in the test solution described later. Therefore, even in the case where it is necessary to put a plurality of reagents are used at high concentrations as in the case of quantifying an enzyme activity, the measurement accuracy can be improved and the test time can be shortened. Furthermore, handling is possible without making the detection unit 5 larger.

That is, since the test solution can be used as a reaction field, the possibility can be reduced that reagents crystallize, precipitate, or something to form scatterers even when the amount of the reagents is large. Therefore, the measurement accuracy can be improved.

In addition, since the test solution can be used as a reaction field, the influence of the diffusion rate of the reaction product changing with the viscosity of a solvent and the like can be suppressed. Therefore, the diffusion rate of the reaction product can be increased, and thereby the time of the test for the enzyme activity can be greatly shortened.

Furthermore, since the detection unit 5 contains only the color former, or the color former and the color development reaction accelerator, it is possible to standardize the test element 1 for certain enzymes to be measured.

Next, a test kit according to the embodiment will now be illustrated.

The test kit according to the embodiment may be a kit that separately includes the test element 1 described above and a test solution containing at least a substrate for an enzyme to be measured and a buffer solution. Also a kit is possible in which the components (e.g. the substrate for an enzyme to be measured, reagents used during performing elementary reactions, the buffer solution, etc.) of the test solution are separately combined and included in place of the test solution. For example, also a kit is possible that separately includes the test element 1 described above, the substrate for an enzyme to be measured, and the buffer solution. Furthermore, the test kit may further include the reagents used during performing elementary reactions.

The buffer solution is used in order to dissolve the substrate for an enzyme to be measured and the reagents used during performing elementary reactions. In this case, the buffer solution is preferably not an organic solvent from the viewpoint of suppressing a decrease in the activity of the reagents and the like. The buffer solution, however, may contain an organic solvent to the extent that the activity of the reagents and the like are kept.

Furthermore, the buffer solution is preferably what can suppress the variation of the hydrogen ion exponent (pH).

In this case, the hydrogen ion exponent of the buffer solution may be within a range from pH 3.5 to 10.0, preferably from pH 4.5 to 9.0.

The buffer solution may be, for example, an acetate buffer, a phosphate buffer, a glycine buffer, a tris buffer, various Good's buffers, and the like.

For example, the buffer solution may be a potassium hydrogen phthalate/sodium hydroxide buffer, disodium citrate/hydrochloric acid buffer, potassium dihydrogen citrate/sodium hydroxide buffer, succinic acid/sodium tetraborate buffer, potassium hydrogen citrate/sodium tetraborate buffer, disodium hydrogen phosphate/citric acid buffer, sodium acetate/hydrochloric acid buffer, acetic acid/sodium acetate buffer, and the like.

The test solution may be also a buffer solution in which the reagents used during performing elementary reactions are further dissolved in addition to the substrate for an enzyme to be measured. That is, the test solution may further contain the reagents used during performing elementary reactions.

In the case where substances other than the enzyme to be measured contained in a sample liquid make elementary reactions excessively vigorous or inhibit elementary reactions, an additive for minimizing the influence may be further added.

In this case, the reagents used during performing elementary reactions may be appropriately selected in accordance with the enzyme to be measured.

Table 1 illustrates substrates for enzymes to be measured, reagents used during performing elementary reactions, color formers, and color development reaction accelerators.

TABLE 1

| | | Enzyme to be measured | | | |
|---|---|---|---|---|---|
| | | ALT(GPT) | AST(GOT) | GGT(γ-GTP) | LDH |
| Test | Substrate | L-alanine + | L-aspartic acid + | L-γ-glutamyl- | L-lactic acid + |
| Solution | | α-ketoglutaric | α-ketoglutaric | L-glutamic | NAD⁺ |
| | | acid | acid | acid + | |
| | | | | glycylglycine | |
| | Product1 | Pyruvic | Oxaloacetic | L-glutamic | — |
| | | acid | acid | acid | |
| | Reagent1 | — | Oxaloacetate | — | — |
| | | | decarboxylase | | |
| | Product2 | — | Pyruvic acid | — | — |
| | Reagent2 | Pyruvate | Pyruvate | L-glutamate | — |
| | | oxidase | oxidase | oxidase | |
| | Product3 | H₂O₂ | H₂O₂ | H₂O₂ | NADH |
| Detection | Color former | TMBZ | TMBZ | TMBZ | Tetrazolium |
| unit | | | | | salt |
| | Color | POD | POD | POD | Diaphorase |
| | development | | | | |
| | reaction | | | | |
| | accelerator | | | | |

FIG. 2 is a schematic diagram for illustrating the way of a reaction.

FIG. 2 illustrates, as an example, the case where the reaction product is produced through a three-step reaction.

When a sample liquid containing an enzyme to be measured is introduced into the test solution, elementary reactions proceed successively from the upper stage of Table 1 to produce the reaction product (in the illustration of Table 1 and FIG. 2, a product 3).

That is, the enzyme to be measured and substrates for the enzyme to be measured react to produce a product 1. Next, the produced product 1 and a reagent 1 react to produce a product 2. Next, the produced product 2 and a reagent 2 react to produce the product 3 (the reaction product). The color former develops a color due to the produced product 3 (the reaction product). At this time, the color development reaction accelerator promotes the color development of the color former.

For example, in the case where hydrogen peroxide (H₂O₂) is produced as the reaction product, the produced hydrogen peroxide (H₂O₂) and a peroxidase (POD) react to produce a radical oxygen atom (O*). The color former is oxidized by the produced radical oxygen atom (O*), for example, the —NH₂ group of TMBZ is oxidized into an ═NH group, to develop blue-green, and further becomes insoluble to precipitate on the surface of the detection unit 5.

By measuring the magnitude of the absorbance change caused by the color development, the enzyme activity can be quantified.

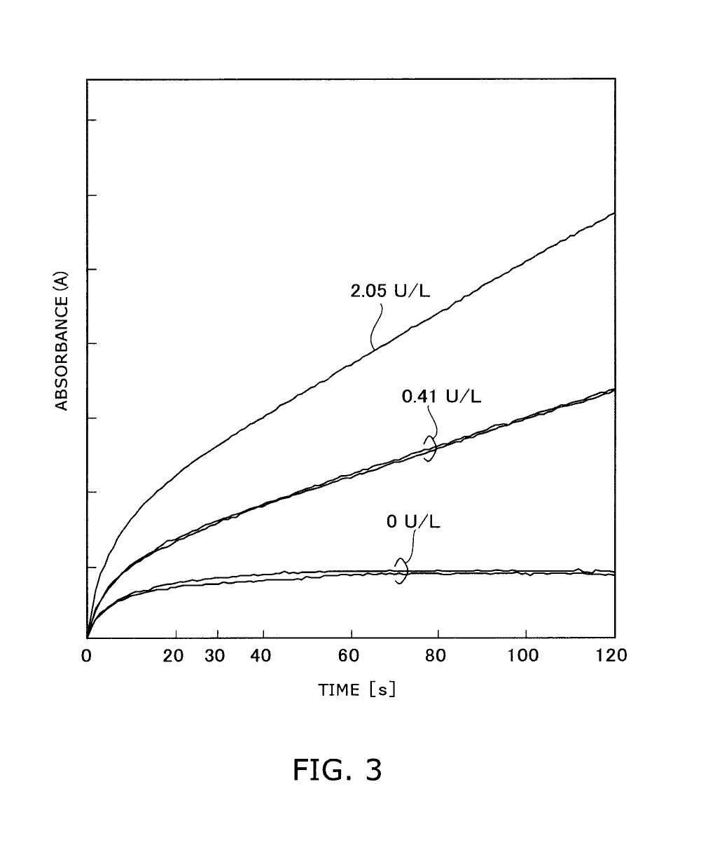

FIG. 3 is a schematic graph for illustrating the change over time of the absorbance.

In the illustration of FIG. 3, L-alanine and α-ketoglutaric acid, which are substrates for ALT (GPT), in a final concentration of 200 mM and pyruvate oxidase, which is a reagent used during performing elementary reactions, in a final concentration of 10 mM were added to a test solution of a commercially available reference serum (Liquid Abnormal; DENKA SEIKEN Co., Ltd.) diluted 20 or 100 times with a 0.2 M phosphate buffer (pH 6.2). Mixing was performed so that the enzyme activity might be 2 U/L and 0.4 U/L, and the mixtures were used. Also a test solution with an enzyme activity of 0 U/L (a test solution of a blank) was used, which is a test solution using a physiological saline solution in place of the serum in the same amount.

As can be seen from FIG. 3, the rate of change in the absorbance of the test solution with an enzyme activity of 0 U/L becomes almost constant about 30 seconds after supplying the test solution to the holding space 6a.

In view of this, the relationship between the magnitude of the absorbance change ΔA and the enzyme activity (ALT (GPT) activity) was found from the difference between the absorbance 30 seconds after the test solution supply and the absorbance 60 seconds after the test solution supply.

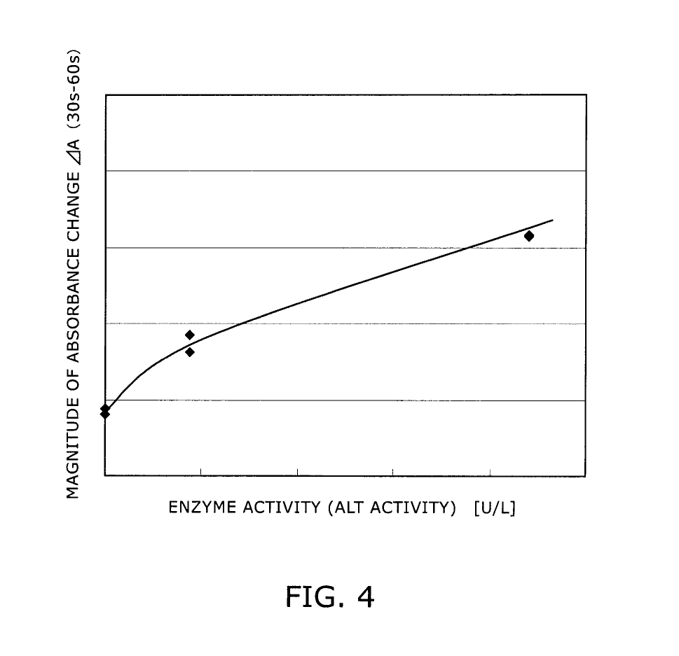

FIG. 4 is a schematic graph for illustrating the relationship between the enzyme activity (ALT (GPT) activity) and the magnitude of the absorbance change.

As shown in FIG. 4, there is a certain relationship between the enzyme activity (ALT (GPT) activity) and the magnitude of the absorbance change. Hence, the enzyme activity can be quantified by measuring the magnitude of the absorbance change caused by the color development. In this case, the relationship between the enzyme activity and the magnitude of the absorbance change in each enzyme may be obtained beforehand for use as a calibration curve. Thereby, the enzyme activity in each enzyme can be easily quantified from the measured value of the magnitude of the absorbance change.

Furthermore, since the magnitude of the absorbance change can be found from the difference between the absorbance 30 seconds after the test solution supply and the absorbance 60 seconds after the test solution supply, the enzyme activity can be quantified in about 60 seconds. Thus, the time of the test for the enzyme activity can be greatly shortened.

According to the embodiment, the substrate for an enzyme to be measured and the reagents can be put in the test solution. Therefore, even in the case where it is necessary to use a plurality of reagents at high concentrations as in the case of quantifying an enzyme activity, the measurement accuracy can be improved and the test time can be shortened. Furthermore, handling is possible without making the detection unit 5 larger.

That is, since the test solution can be used as a reaction field, the possibility can be reduced that reagents crystallize, precipitate, or something to form scatterers even when the amount of the reagents is large. Therefore, the measurement accuracy can be improved.

In addition, since the test solution can be used as a reaction field, the influence of the diffusion rate of the reaction product changing with the viscosity of a solvent and the like can be suppressed. Therefore, the diffusion rate of the reaction product can be increased, and thereby the time of the test for the enzyme activity can be greatly shortened.

Furthermore, since the detection unit 5 contains only the color former, or the color former and the color development reaction accelerator, it is possible to standardize the test element 1 for certain enzymes to be measured.

Next, a test device according to the embodiment will now be illustrated.

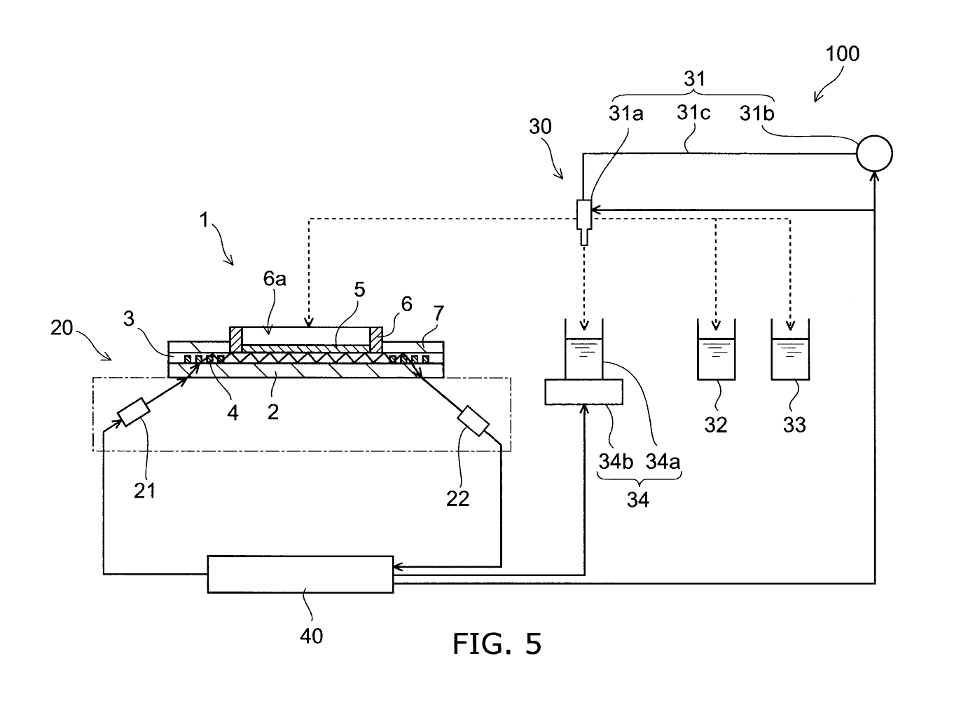

FIG. 5 is a schematic view for illustrating a test device according to the embodiment.

As shown in FIG. 5, a test device 100 includes a test unit 20, a solution reaction unit 30, and a control unit 40.

In the test unit 20, the test element 1 described above can be attached and removed.

In this case, the test element 1 may be used only once and then thrown away, and may be exchanged for each test.

The test unit 20 includes a light sending unit 21 and a light receiving unit 22. More specifically, the test unit 20 includes the light sending unit 21 that sends light into one optical element unit 4 provided in the test element 1 and the light receiving unit 22 that receives the light from the other optical element unit 4 and converts it into an electric signal in accordance with the intensity of the light. The light sending unit 21 may be, for example, a laser diode or the like. The light receiving unit 22 may be, for example, a photodiode or the like.

The light sending unit 21 is provided in a position whereby light can enter one optical element unit 4. The light receiving unit 22 is provided in a position where the light from the other optical element unit 4 can be received.

Here, the light emitted from the light sending unit 21 and having entered the optical element unit 4 via the base 2 is diffracted at the interface between the optical element unit 4 and the optical waveguide unit 3, and then propagates while being reflected multiple times at the interfaces between the optical waveguide unit 3, and the base 2 and the detection unit 5. The light having thus entered is transmitted being reflected in the optical waveguide unit 3, and at this time an evanescent wave is generated. The evanescent wave refers to an electromagnetic wave that is generated at the interface between the optical waveguide unit 3 and the exterior and is transmitted only through the surface when the light is totally reflected at the interface. The distance the evanescent wave reaches is about the wavelength (1 μm or less) from the surface of the optical waveguide unit 3.

When the evanescent wave is refracted at the interface between the optical waveguide unit 3 and the detection unit 5, the evanescent wave is absorbed in accordance with the magnitude of the absorbance change caused by the color development of the color former in the detection unit 5 described above.

The light having thus propagated in the optical waveguide unit 3 is emitted outward from the other optical element unit 4 via the base 2. The light emitted outward is received by the light receiving unit 22 and is converted into an electric signal in accordance with the intensity of the received light. The converted electric signal is sent to the control unit 40.

In this case, the intensity of the light received by the light receiving unit 22 is a value having changed in accordance with the magnitude of the absorbance change caused by the color development of the color former in the detection unit 5 (a value having changed in accordance with the absorption of the evanescent wave). Therefore, the enzyme activity can be quantified from the rate of the change or the like.

The solution reaction unit 30 includes a supply unit 31, a solution storage unit 32, a sample liquid storage unit 33, and a mixing unit 34.

In the supply unit 31, a nozzle 31a is provided and an aspiration unit 31b such as a pump is connected to the nozzle 31a via a flexible tube 31c. The position of the nozzle 31a can be changed by a not-shown movement unit. In this case, by moving the nozzle 31a as shown by the broken lines in the drawing, the aspiration and discharge of a liquid such as the test solution can be performed in the solution storage unit 32, the sample liquid storage unit 33, the mixing unit 34, and the holding space 6a.

The solution storage unit 32 stores the test solution described above. For example, the solution storage unit 32 stores a buffer solution in which the substrate for an enzyme to be measured is dissolved, a buffer solution in which the substrate for an enzyme to be measured and the reagents used during performing elementary reactions are dissolved, and the like.

The sample liquid storage unit 33 stores a sample liquid containing an enzyme to be measured. For example, the sample liquid storage unit 33 stores a sample liquid such as diluted blood or serum.

The mixing unit 34 includes a storage unit 34a and a stirring unit 34b.

A prescribed amount of the test solution and the sample liquid are supplied to the storage unit 34a by the supply unit 31, and the test solution and the sample liquid are stirred by the stirring unit 34b.

That is, the mixing unit 34 mixes at least the substrate for the enzyme to be measured, the buffer solution, and the sample liquid containing the enzyme.

The control unit 40 controls the operation of the components provided in the test unit 20 and the solution reaction unit 30. Furthermore, the control unit 40 quantifies the enzyme activity based on the electric signal sent from the light receiving unit 22.

In this case, the absorbance A after a prescribed time can be calculated by Formula (I) below, and further the magnitude of the absorbance change ΔA can be calculated. For example, the absorbance A(30) 30 seconds after the test solution supply and the absorbance A(60) 60 seconds after the test solution supply can be calculated by Formula (I), and further the magnitude of the absorbance change AA can be calculated from the difference between the absorbance A(30) and the absorbance A(60).

Further, as described above, the enzyme activity can be found from the magnitude of the absorbance change LA based on a calibration curve obtained beforehand and the like.

A(t1)=−log(I(t1)/I(t0)) (1)

where A(t1) is the absorbance after t1 seconds assuming that t=t0 is the initial value, I(t0) is the light detection intensity (e.g. the output value of the light receiving unit 22) at time t0, and I(t1) is the light detection intensity (e.g. the output value of the light receiving unit 22) after t1 seconds assuming that t=t0 is the initial value.

Next, an operation of the test device 100 will now be illustrated.

First, the test solution is supplied to the solution storage unit 32 and a sample liquid such as diluted blood and serum is supplied to the sample liquid storage unit 33, by a worker or the like. The test element 1 is attached to the test unit 20.

Next, the nozzle 31a is moved using the not-shown movement unit to put the tip of the nozzle 31a into the test solution in the solution storage unit 32. Then, the aspiration unit 31b is operated to aspirate a prescribed amount of the test solution.

Next, the nozzle 31a is moved using the not-shown movement unit to put the tip of the nozzle 31a into the storage unit 34a. Then, the aspiration unit 31b is operated to discharge the test solution into the storage unit 34a.

Next, the nozzle 31a is moved using the not-shown movement unit to put the tip of the nozzle 31a into the sample liquid in the sample liquid storage unit 33. Then, the aspiration unit 31b is operated to aspirate a prescribed amount of the sample liquid.

Next, the nozzle 31a is moved using the not-shown movement unit to put the tip of the nozzle 31a into the storage unit 34a. Then, the aspiration unit 31b is operated to discharge the sample liquid into the storage unit 34a.

When the prescribed amount of the test solution and the sample liquid are supplied into the storage unit 34a, the elementary reactions of the enzyme contained in the sample liquid with the substrate for the enzyme to be measured and the reagents contained in the test solution proceed to produce the reaction product. At this time, by using the stirring unit 34b to stir the test solution and the sample liquid, the progress of the elementary reactions is promoted.

Next, the nozzle 31a is moved using the not-shown movement unit to put the tip of the nozzle 31a into the liquid in the storage unit 34a. Then, the aspiration unit 31b is operated to aspirate a prescribed amount of the liquid. At this stage, the produced reaction product is contained in the liquid.

Next, the nozzle 31a is moved using the not-shown movement unit to position the tip of the nozzle 31a immediately above the holding space 6a. Then, the aspiration unit 31b is operated to discharge the liquid containing the reaction product into the holding space 6a. The color former provided in the detection unit 5 develops a color due to the reaction product contained in the liquid discharged into the holding space 6a. At this time, in the case where the color development reaction accelerator is contained, the color development reaction accelerator promotes the color development of the color former.

Next, light such as a laser beam is emitted from the light sending unit 21, and is made to be reflected and transmitted in the optical waveguide unit 3 to generate an evanescent wave. When the evanescent wave is refracted at the interface between the optical waveguide unit 3 and the detection unit 5, the evanescent wave is absorbed in accordance with the magnitude of the absorbance change caused by the color development of the color former in the detection unit 5 described above.

The light having propagated in the optical waveguide unit 3 is emitted outward from the other optical element unit 4 via the base 2, and is received by the light receiving unit 22. The intensity of the light received by the light receiving unit 22 is a value having changed in accordance with the magnitude of the absorbance change caused by the color development of the color former in the detection unit 5. Thus, the enzyme activity is quantified from the rate of the change or the like.

In the case of the foregoing, the case is illustrated where a buffer solution in which the substrate for an enzyme to be measured is dissolved or a buffer solution in which the substrate for an enzyme to be measured and the reagents used during performing elementary reactions are dissolved is stored in the solution storage unit 32. However, the embodiment is not limited thereto. For example, a configuration is also possible in which each of the substrate for an enzyme to be measured and the reagents used during performing elementary reactions is dissolved in a buffer solution; the resulting buffer solutions are separately stored; and the supply unit 31 supplies the test solutions individually to the storage unit 34a.

Furthermore, the solution reaction unit 30 is not necessarily needed. For example, a worker may mix the test solution described above and a sample liquid and supply the liquid containing the produced reaction product into the holding space 6a.

Furthermore, the control unit 40 is not necessarily needed. For example, the electric signal sent from the light receiving unit 22 may be operated with a separately provided arithmetic means or the like.

Moreover, a washing means that washes the interior of the nozzle 31a, the interior of the flexible tube 31c, and the like may be provided as appropriate. For example, it is possible to aspirate wash water etc. to wash the interior of the nozzle 31a, the interior of the flexible tube 31c, and the like. It is also possible to run wash water etc. into the flexible tube 31c to wash the interior of the nozzle 31a, the interior of the flexible tube 31c, and the like.

According to the embodiment, the substrate for an enzyme to be measured and the reagents can be put in the test solution. Therefore, even in the case where it is necessary to use a plurality of reagents at high concentrations as in the case of quantifying an enzyme activity, the measurement accuracy can be improved and the test time can be shortened. Furthermore, handling is possible without making the detection unit 5 larger.

That is, since the test solution can be used as a reaction field, the possibility can be reduced that reagents crystallize, precipitate, or something to form scatterers even when the amount of the reagents is large. Therefore, the measurement accuracy can be improved.

In addition, since the test solution can be used as a reaction field, the influence of the diffusion rate of the reaction product changing with the viscosity of a solvent and the like can be suppressed. Therefore, the diffusion rate of the reaction product can be increased, and thereby the time of the test for the enzyme activity can be greatly shortened.

Furthermore, since the detection unit 5 contains only the color former, or the color former and the color development reaction accelerator, it is possible to standardize the test element 1 for certain enzymes to be measured.

Next, a test method according to the embodiment will now be illustrated.

The test method according to the embodiment can use the test element 1, the test kit, and the test device 100 described above.

Herein, the case of using the test kit described above is illustrated as an example.

First, the test solution described above is dispensed.

In this case, the test solution may be a buffer solution in which at least the substrate for an enzyme to be measured is dissolved. Also, the test solution may be a buffer solution in which the reagents used during performing elementary reactions are further dissolved in addition to the substrate for an enzyme to be measured. In the case where substances other than the enzyme to be measured contained in a sample liquid make elementary reactions excessively vigorous or inhibit elementary reactions, an additive for minimizing the influence may be further added.

In the case of a kit in which the components (e.g. the substrate for an enzyme to be measured, the reagents used during performing elementary reactions, the buffer solution, etc.) of the test solution are separately combined and included in place of the test solution, these are used to prepare the test solution.

Next, a sample liquid is introduced into the test solution.

The sample liquid may be, for example, blood, serum, or the like in which an enzyme to be measured is contained and which is diluted a prescribed times.

When the sample liquid is introduced into the test solution, elementary reactions proceed to produce the reaction product.

In this case, by stirring after introducing the sample liquid into the test solution, the progress of the elementary reactions can be promoted.

Next, the liquid containing the reaction product is supplied in a prescribed amount into the holding space 6a of the test element 1. The color former provided in the detection unit 5 develops a color due to the reaction product. At this time, in the case where the color development reaction accelerator is contained, the color development reaction accelerator promotes the color development of the color former.

Next, the magnitude of the absorbance change caused by the color development of the color former is found, and the enzyme activity is found from the magnitude of the absorbance change based on a calibration curve obtained beforehand and the like.

The magnitude of the absorbance change can be found by, for example, using the test unit 20 described above to detect the light detection intensity to find the absorbance, and using the difference in the absorbance at a prescribed time.

That is, a method is possible in which at least the substrate for an enzyme to be measured, the buffer solution, and a sample liquid containing the enzyme are mixed; the mixed liquid is supplied in a prescribed amount into the holding unit 6 provided in the test element 1; the magnitude of the absorbance change caused by the color development of the color former provided in the detection unit 5 of the test element 1 is found; and the enzyme activity is found based on the magnitude of the absorbance change.

The calculation of the magnitude of the absorbance change, the method for finding the enzyme activity from the magnitude of the absorbance change based on a calibration curve, and the like are similar to those described above, and a detailed description is therefore omitted.

According to the embodiment, the substrate for an enzyme to be measured and the reagents can be put in the test solution. Therefore, even in the case where it is necessary to use a plurality of reagents at high concentrations as in the case of quantifying an enzyme activity, the measurement accuracy can be improved and the test time can be shortened.

That is, since the test solution can be used as a reaction field, the possibility can be reduced that reagents crystallize, precipitate, or something to form scatterers even when the amount of the reagents is large. Therefore, the measurement accuracy can be improved.

In addition, since the test solution can be used as a reaction field, the influence of the diffusion rate of the reaction product changing with the viscosity of a solvent and the like can be suppressed. Therefore, the diffusion rate of the reaction product can be increased, and thereby the time of the test for the enzyme activity can be greatly shortened.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

For example, the shape, dimensions, material, arrangement, number, and the like of the components of the test element 1, the test kit, and the test device 100 are not limited to those illustrated but may be altered as appropriate.

What is claimed is:

1. A test kit comprising:
a test element; and
a test solution containing a substrate for an enzyme to be measured and a buffer solution,
the test element including:
a base having transparency;
a pair of optical element units arranged away from each other on a major surface of the base;
an optical waveguide unit provided on the major surface of the base;
a detection unit provided on a major surface of the optical waveguide unit of between the optical element units, the major surface of the optical waveguide being an opposite side which touches the base; and
a holding unit in a frame shape, one end of the holding unit being provided to protrude from a major surface of the detection unit,
the detection unit including a color former and a film-formed body holding the color former.

2. The test kit according to claim 1, wherein the test solution further contains a reagent used during performing an elementary reaction.

3. The test kit according to claim 1, wherein the test solution further contains an additive controlling an elementary reaction.

4. The test kit according to claim 1, wherein a hydrogen ion exponent of the buffer solution is not less than pH 3.5 and not more than pH 10.0.

5. A test kit comprising:
a test element;
a substrate for an enzyme to be measured; and
a buffer solution,
the test element including:
a base having transparency;
a pair of optical element units arranged away from each other on a major surface of the base;
an optical waveguide unit provided on the major surface of the base;
a detection unit provided on a major surface of the optical waveguide unit of between the optical element units, the major surface of the optical waveguide being an opposite side which touches the base; and
a holding unit in a frame shape, one end of the holding unit being provided to protrude from a major surface of the detection unit,
the detection unit including a color former and a film-formed body holding the color former.

6. The test kit according to claim 5, further comprising a reagent used during performing an elementary reaction.

7. The test kit according to claim 5, further contains an additive controlling an elementary reaction.

8. The test kit according to claim 5, wherein a hydrogen ion exponent of the buffer solution is not less than pH 3.5 and not more than pH 10.0.

9. A test method comprising:
mixing at least a substrate for an enzyme to be measured, a buffer solution, and a sample liquid containing the enzyme;
supplying the mixed liquid in a prescribed amount into a holding unit provided in a test element;
finding a magnitude of an absorbance change caused by color development of a color former provided in a detection unit of the test element; and
finding an enzyme activity based on the magnitude of the absorbance change,
the test element including:
a base having transparency;
a pair of optical element units arranged away from each other on a major surface of the base;
an optical waveguide unit provided on the major surface of the base;
the detection unit provided on a major surface of the optical waveguide unit of between the optical element units, the major surface of the optical waveguide being an opposite side which touches the base; and
the holding unit in a frame shape, one end of the holding unit being provided to protrude from a major surface of the detection unit,
the detection unit including a color former and a film-formed body holding the color former.

10. The test method according to claim 9, wherein,
in the finding the magnitude of the absorbance change, an absorbance is calculated at a prescribed time interval and the magnitude of the absorbance change is calculated based on the calculated absorbance and,
in the finding the enzyme activity, the enzyme activity is found from a calibration curve obtained beforehand and the calculated magnitude of the absorbance change.

11. The test method according to claim 10, wherein the calculation of the absorbance is made using a formula below:
A(t1)=−log(I(t1)/I(t0))
where A(t1) is an absorbance after t1 seconds assuming that t=t0 is an initial value, I(t0) is a light detection intensity at time to, and I(t1) is a light detection intensity after t1 seconds assuming that t=t0 is an initial value.

12. The test method according to claim 9 including:
sending light into one of the pair of optical element units provided in the test element;
reflecting and transmitting the light having entered in the optical waveguide unit provided in the test element to generate an evanescent wave; and
absorbing the evanescent wave in accordance with the magnitude of the absorbance change caused by the color development of the color former.
